(19) United States
(12) Patent Application Publication
(10) Pub. No.: US 2020/0261258 A1
(43) Pub. Date: Aug. 20, 2020

(54) DEVICES, SYSTEMS, METHODS AND KITS FOR INSERTION AND REMOVAL OF IUDS

(71) Applicant: MEDICINES360, San Francisco, CA (US)

(72) Inventors: Rob DECKMAN, San Bruno, CA (US); Mitchell David CREININ, Davis, CA (US); Jessica Dorothy GROSSMAN, Tiburon, CA (US); Andrea Ildiko OLARIU, Redwood City, CA (US)

(73) Assignee: MEDICINES360, San Francisco, CA (US)

(21) Appl. No.: 16/793,354

(22) Filed: Feb. 18, 2020

Related U.S. Application Data
(60) Provisional application No. 62/807,373, filed on Feb. 19, 2019.

(51) Int. Cl. A61F 6/18

(52) U.S. Cl. CPC A61F 6/18

(57) ABSTRACT

Systems, methods and kits for self-insertion and self-removal of an IUD device. Devices include a handle

[symbol]

### CROSS-REFERENCE

This application claims the benefit of U.S. Provisional Application No. 62/807,373, filed Feb. 19, 2019, entitled DEVICES, SYSTEMS AND METHODS FOR INSERTION AND REMOVAL OF IUDS which application is incorporated herein in its entirety by reference.

### BACKGROUND

The rates of unwanted pregnancies in developing and developed countries have continued to decrease over the last 10 years. However, barriers to accessing contraception is still a significant issue for jurisdictions where access to healthcare is limited. According to the World Health Organization (WHO) additional barriers to access contraception can be based on age, marital status, health worker bias, and/or lack of willingness to acknowledge sexual health needs. Knowledge, transportation and financial constraints are also significant barriers to access.

Improving access to contraception continues to be an important objective for a variety of reasons. Adolescent pregnancy remains a major contributor to maternal and child mortality and to intergenerational cycles of ill-health and poverty. Although there are several types of contraception available, the intrauterine device (IUD) continues to be a reliable, easy to deploy, low cost option for preventing unplanned pregnancies. However, accessibility to the IUD as a contraceptive method for women living in underserved communities continues to be a challenge due to lack of access to a medical clinic or physician.

What is needed are methods, devices, systems and kits that can be used for IUD insertion or removal by a patient or a patient and a partner in a remote location when access to a medical clinic or physician is not readily available.

### SUMMARY

Disclosed are methods, devices, systems and kits that can be used for IUD insertion or removal by a patient or a patient and a partner in a remote location when access to a medical clinic or a physician is not available. Partners can include, for example, local health care promoters, nurse practitioners, friends or relatives.

An aspect of the disclosure is directed to IUD self-insertion devices. The IUD self-insertion devices comprise: an elongated handle with a knob at a proximal end and a control slider moveable in a channel along a length of the handle; a hollow shaft positioned at a distal end of the elongated handle; a plunger positioned within the handle and the hollow shaft having a plurality of teeth along at least a portion of an outer surface of the plunger; two or more arms positioned at a distal end of the hollow shaft wherein the arms are rotatable away from a central axis of the hollow shaft; a ratchet member positioned on an interior surface of the handle configured to engage the plurality of teeth along the outer surface of the plunger; one or more tools positioned on an interior surface of at least one of the arms; and a power source in communication with the one or more tools. Additionally, the control slider can be a bilaterally controllable slider with a first slider interface moveable within a first channel and a second slider interface moveable within a second channel. In some configurations, the elongated handle has a curved exterior surface. Additionally, the knob is configurable to move in a proximal and distal direction. Additionally, the plunger is configurable to float within the interior of the self-insertion device. Suitable tools for the devices are selected from a light source and a camera. Additionally, the visual tool arms have a curved recess in an outer surface at the distal end that holds arms of an IUD prior to loading the IUD into the insertion device wherein the IUD has a t-shape and comprises an elongated member and a pair of IUD arms. The elongated member of the IUD can further comprise a core part around which a jacket-like polymeric reservoir containing an active agent has been fitted. The active agent can be a hormone used for the treatment of menopausal troubles or for contraception.

Another aspect of the disclosure is directed to IUD self-removal devices. Suitable IUD self-removal devices comprise: an elongated handle with a push rod moveable within an interior of the handle; a cup comprising a wall with an inner wall surface defining a cavity and an outer wall surface, an open end and a closed end, a stem extending from an exterior location on the wall opposite the open end wherein the stem is configurable to engage a distal end of the push rod. A vacuum release rod can also be provided. In some configurations, a rib on an interior surface of the elongated handle.

Still another aspect of the disclosure is directed to IUD self-removal devices comprising: an elongated handle forming an elongated shaft; a push rod positioned within the elongated handle having a connector at a distal end of the push rod; and a grabber comprising two or more petals positioned at a distal end of the push rod wherein the petals are rotatable away from a central axis of the push rod wherein an interior surface of the two or more petals have ridges. Additionally, the devices can comprise one or more tools positioned on an interior surface of at least one of the petals. A power source can also be provided which is in communication with the one or more tools. Suitable tools include, for example, a light source and a camera. The devices can also have a curved exterior surface along at least a portion of the handle. The push rod can also be configurable to move in a proximal and distal direction.

Yet another aspect of the disclosure is directed to IUD self-removal devices comprising: an elongated handle with a push rod having a distal end and a proximal end wherein the push rod is moveable in a distal direction and a proximal direction within an interior of the handle; at least one of bristles positioned at the distal end of the push rod, a plurality of threads positioned at the distal end of the push rod, a lasso positioned at the distal end of the push rod, and a hook positioned at the distal end of the push rod. The devices can further comprise one or more tools positioned on a surface of the one or more petals. Suitable tools include, for example, light source and a camera.

Another aspect of the disclosure is directed to kits. Kits can comprise: at least one of a self-insertion device with an IUD and a self-removal device; and at least one of a vaginal cavity depth measurement device; a battery; forceps; a visualization cone; and a self-removal verification card. The visualization cone can include a forceps guide positioned on a surface. In some configurations the visualization cone is self-illuminating.

Still another aspect of the disclosure is directed to methods of inserting an IUD into a patient. The methods comprise the steps of: providing a self-insertion device comprising an elongated handle with a knob at a proximal end and a control slider moveable in a channel along a length of the handle, a hollow shaft positioned at a distal end of the elongated handle, a plunger positioned within the handle and the hollow shaft having a plurality of teeth along at least a portion of an outer surface of the plunger, two or more arms positioned at a distal end of the hollow shaft wherein the arms are rotatable away from a central axis of the hollow shaft, a ratchet member positioned on an interior surface of the handle configured to engage the plurality of teeth along the outer surface of the plunger, one or more tools positioned on an interior surface of at least one of the arms, and a power source in communication with the one or more tools; inserting the self-insertion device into a vaginal cavity; pulling the knob of the self-insertion device in a distal direction; viewing a cervix; advancing the inner delivery tube into the cervix; releasing the IUD from the self-insertion device; and removing the self-insertion device.

Another aspect of the disclosure is directed to methods of inserting an IUD into a patient of claim 25, further comprising the step of: measuring a depth of the vaginal cavity. Methods can further comprising the steps of: acquiring an image of the cervix; and transmitting the image of the cervix to a location remote from the patient.

Yet another aspect of the disclosure is directed to methods of removing an inserted IUD from a patient comprising the steps of: providing a self-removal device comprising an elongated handle with a push rod moveable within an interior of the handle, a cup comprising a wall with an inner wall surface defining a cavity and an outer wall surface, an open end and a closed end, a stem extending from an exterior location on the wall opposite the open end wherein the stem is configurable to engage a distal end of the push rod; inserting the self-removal device into a vaginal cavity; positioning the cup near the cervix; creating a suction around an IUD removal thread; pulling the cup in a proximal direction; and removing the cup and self-insertion device. The methods can further include the step of the using a vacuum release rod to release a vacuum of the cup against the cervix. Additional steps include one or more of measuring a depth of the vaginal cavity, acquiring an image of the cervix, and transmitting the image of the cervix to a location remote from the patient.

Another aspect of the disclosure is directed to methods of removing an inserted IUD from a patient comprising the steps of: providing a self-removal device comprising an elongated handle forming an elongated shaft, a push rod positioned within the elongated handle having a connector at a distal end of the push rod, and a grabber comprising two or more petals positioned at a distal end of the push rod wherein the petals are rotatable away from a central axis of the push rod wherein an interior surface of the two or more petals have ridges; inserting the self-removal device into a vaginal cavity; positioning the petals near the cervix; pushing the push rod in a distal direction to extend the petals away from a central axis; pulling the push rod in a proximal direction to retract the petals towards the central axis to engage the IUD threads; and removing the self-removal device. Additionally, the methods can include one or more steps of measuring a depth of the vaginal cavity, acquiring an image of the cervix, and transmitting the image of the cervix to a location remote from the patient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

U.S. Pat. No. 2,534,900 A to Chalmers for Menstrual Cup issued Dec. 19, 1950;

U.S. Pat. No. 3,407,806 A to Hulka et al. for Contraceptive Intra-Uterine Devices issued Oct. 29, 1968;

U.S. Pat. No. 3,783,861 A to Abramson for Inserter for Intrauterine Devices issued Jan. 8, 1974;

U.S. Pat. No. 3,794,025 A to Lerner for Intrauterine Device Saddle Inserter issued Feb. 26, 1974;

U.S. Pat. No. 3,902,483 A to Place et al. for Intrauterine Device with Locator Means for Indicating Uterine Position of Device issued Sep. 2, 1975;

U.S. Pat. No. 3,937,217 A to Kosonen for Intrauterine Contraceptive Device issued Feb. 10, 1976;

U.S. Pat. No. 4,353,363 A to Quesada for Intrauterine Spermicide issued Oct. 12, 1982;

U.S. Pat. No. 4,359,046 A to Shaw Jr. for IUD Arrangement issued Nov. 16, 1982;

U.S. Pat. No. 4,372,302 A to Akerlund for Instrument for Retrieval of Retracted Threads of Intrauterine Contraceptive Devices issued Feb. 8, 1983;

U.S. Pat. No. 4,381,001 A to Shaw Jr. for IUD Arrangement issued Apr. 26, 1983;

U.S. Pat. No. 4,495,934 A to Shaw Jr. for IUD Arrangement issued Jan. 29, 1985;

U.S. Pat. No. 4,830,025 A to Gainutdinova et al. for Intrauterine Contraceptive Device issued May 16, 1989;

U.S. Pat. No. 4,920,727 A to Ristimaki et al. for Cassette System and Apparatus for Manufacturing an Active Agent Liberating Capsule for Subcutnaeous Use issued May 1, 1990;

U.S. Pat. No. 4,949,732 A to Spoon et al. for Apparatus for Insertion and Fixation of an Intra Uterine Contraceptive Device to the Uterine Fundus issued Aug. 21, 1990;

U.S. Pat. No. 4,957,119 A to de Nijs for Contraceptive Implant issued Sep. 18, 1990;

U.S. Pat. No. 5,084,004 A to Ranoux for Process for Intra-Uterine Fertilization in Mammals and Device for Implementation Thereof issued Jan. 28, 1992;

U.S. Pat. No. 5,088,505 A to de Nijs for Contraceptive Implant issued Feb. 18, 1992;

U.S. Pat. No. 5,370,129 A to Diaz et al. for IUD Inserting Apparatus issued Dec. 6, 1994;

U.S. Pat. No. 5,400,804 A to Helle et al. for Method and an Equipment for Installing a Medicine Capsule on a Support issued Mar. 28, 1995;

U.S. Pat. No. 5,785,053 A to Macandrew et al. for Inserter for the Positioning of an Intrauterine Device issued Jul. 28, 1998;

U.S. Pat. No. 6,039,968 A to Nabahi for Intravaginal Drug Delivery Device issued Mar. 21, 2000;

U.S. Pat. No. 6,056,976 A to Markkula et al. for Elastomer, Its Preparation and Use issued May 2, 2000;

U.S. Pat. No. 6,063,395 A to Markkula et al. for Drug Delivery Device Especially for the Delivery of Progestins and Estrogens issued May 16, 2000;

U.S. Pat. No. 6,103,256 A to Nabahi for Intravaginal Drug Delivery Device issued Aug. 15, 2000;

U.S. Pat. No. 6,117,442 A to Markkula et al. for Drug Delivery Device, Especially for the Delivery of Androgens issued Sep. 12, 2000;

U.S. Pat. No. 7,862,552 B2 to McIntyre et al. for Medical Devices for Treating Urological and Uterine Conditions issued Jan. 4, 2011;

U.S. Pat. No. 10,028,858 B2 to Deckman et al. for Intrauterine Systems, IUD Insertion Devices, and Related Methods and Kits Therefor issued Jul. 24, 2018;

U.S. Patent Pub. US 2005/0045183 A1 to Callister et al. for Methods and Devices for Occluding Body Lumens and/or for Delivering Therapeutic Agents published Mar. 3, 2005;

U.S. Patent Pub. US 2008/0077097 A1 to Chambers et al. for Menstrual Cup published Mar. 27, 2008; and

U.S. Patent Pub. US 2008/0095825 A1 to LaFont for Method for Making a Reservoir Containing an Active Substance Diffused Through the Reservoir and Installation Therefor published Apr. 24, 2008.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a t-shaped IUD;

FIG. 2 is a t-shaped IUD positioned within a uterus;

FIG. 3 illustrates a self-insertion IUD loading device;

FIGS. 4A-G illustrate a self-insertion IUD loading device at different stages of operation;

FIGS. 5A-B illustrate a perspective view of an insertion device with a camera and a light source;

FIG. 6 illustrates a guidewire IUD insertion device;

FIGS. 7A-C illustrate a rotational IUD insertion device;

FIGS. 8A-B illustrate a t-shaped IUD positioned within the uterus and a finger coated hand removing the IUD by grasping the thread;

FIGS. 9A-C illustrate a finger cup IUD removal device with a hook for grasping an IUD;

FIGS. 10A-J illustrate a suction IUD removal device and a tool for moving the cup into the distal end of the inserter;

FIGS. 11A-B illustrate another suction IUD removal device;

FIGS. 12A-E illustrate a petal-thread grabber IUD removal device;

FIGS. 13A-E illustrate a petal-thread grabber IUD removal device in use;

FIGS. 14A-B illustrate a brush thread grabber IUD removal device;

FIG. 15 illustrates an alternative brush thread grabber IUD removal device;

FIGS. 16A-B illustrate a lasso thread grabber IUD removal device;

FIG. 17 illustrates a kit for an IUD removal device;

FIG. 18 illustrates a disposable kit for removing an IUD; and

FIG. 19 illustrates a self-removal verification card.

### DETAILED DESCRIPTION

Throughout the disclosure and figures the following markings have been used: a axis; 10 proximal or proximal position; and 20 distal or distal position. The proximal position reflects the location nearest the user in use; the distal position reflects the location furthest from the user in use. The axis is an axis about which an action occurs.

The insertion and removal devices disclosed herein are sized to at least partially fit within a female vagina 34 to engage the cervix 32. A portion of the insertion devices are sized so that a distal end can insert a t-shaped IUD into the uterus 30. As will be appreciated by those skilled in the art, although there is variability in the actual length and width of the vagina, the female anatomy can typically be organized into sizes and tools engaging the female anatomy can also be organized into sizes. The average depth of the vagina (measured from the opening to the tip of the cervix) is about 3.77 inches (9.6 cm), and the size range has been reported to be from about 3 inches to 7 inches (7.6 cm to 17.7 cm). The cervix is typically about 1 inch (about 2 cm to 3 cm) in length.

In general, the self-insertion devices and self-removal devices disclosed herein will have an overall dimension in a range of from 3.1 inches (80 mm) to 5.1 inches (130 mm) in length, more preferably 3.5 inches (90 mm) to 4.7 inches (120 mm) in length, and even more preferably 4.1 inches (105 mm) to 4.2 inches (108 mm) in length. The overall diameter of the distal 20 end of the devices are from 0.39 inches (10 mm) to 0.79 inches (20 mm), more preferably from 0.47 inches (12 mm) to 0.71 (18 mm), and even more preferably 0.59 inches (15 mm). The handle of the devices can have a larger diameter than the distal (insertion) end of the devices.

### I. Intrauterine Devices (IUDS)

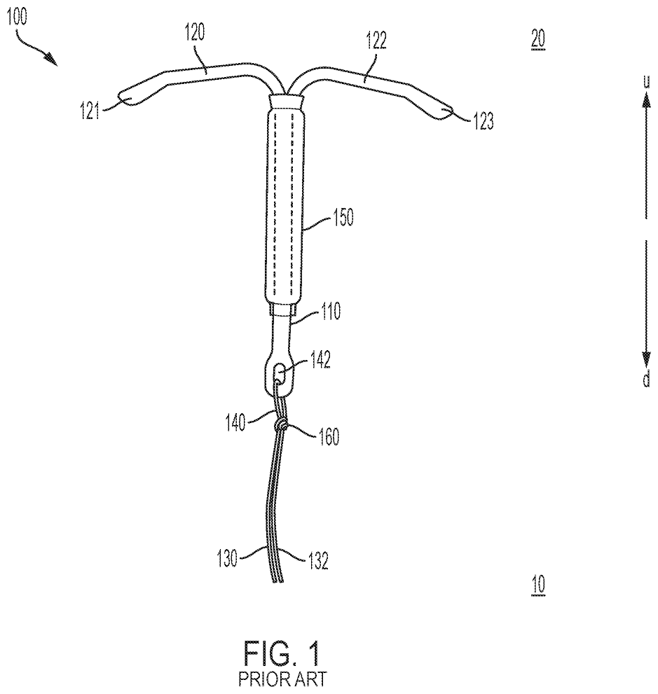

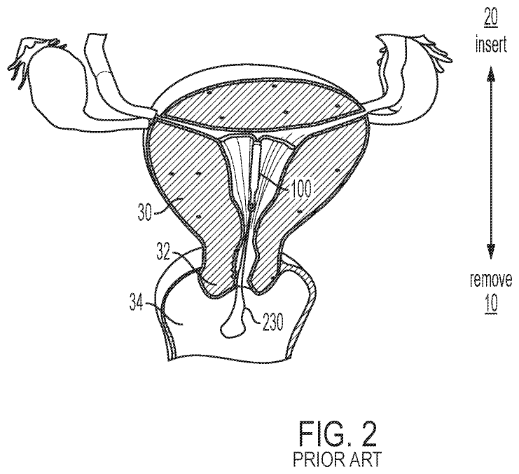

FIG. 1 is a t-shaped IUD 100. IUDs typically have a length of from about 31.90 mm to about 32.22 mm and a width of from about 31.81 mm to about 32.13 mm when the IUD is in a fully deployed position. As will be appreciated by those skilled in the art, the length does not include the knot or removal threads that may accompany the IUD.

The T-shaped IUD 100 comprises an elongated body 110 having a proximal end 10 and a distal end 20. The elongated body 110 can include a coating such as a time-release drug, hormone, or copper. A drug delivery t-shaped IUD typically has four components: the t-shaped IUD, the removal thread at the proximal end of the IUD, a drug reservoir and an exterior membrane surrounding the drug reservoir that enables the drug in the reservoir to be time-released.

The elongated body 110 can be formed from any suitable material, including, but not limited to plastic or copper. At the distal end 20 of the IUD (i.e., the end positioned away from the physician's hand), IUD arms 120, 122 are attached to or integrally formed with the elongated body 110. The IUD arms 120, 122 are configurable to fold upward u or downward d to minimize the IUD cross-section such that the IUD can fit within an interior of an insertion device sheath or tube for insertion through the cervix and into the uterus. Additionally, either or both of the IUD arms 120, 122 are configurable to include an enlarged or bulbous tip 121, 123, which can, for example, have a curved, spherical or semi-spherical shape. The bulbous tip 121, 123 of the IUD arms 120, 122 can be formed such that the IUD arms, when folded upward u and pushed together, form a smooth and rounded distal tip. The IUD is not shipped or stored with the arms folded up to avoid deforming the arms.

At a proximal end 10 of the t-shaped IUD 100, the IUD can further include one or more IUD threads 130, 132 (or strings) attached to the IUD. The IUD threads 130, 132 are connectable to the IUD at a connection point 140, e.g., a single thread with two ends passed through an aperture and tied in a knot as illustrated. The connection point 140 can have an aperture 142 through which the string passes. The IUD thread can also be configured to form a loop through the connection point.

Although the insertion devices are generally described herein with regard to a T-shaped IUD such as the IUD shown in FIG. 1, it should be noted that the insertion devices of the present disclosure are adaptable to facilitate insertion of other IUD configurations, as would be appreciated by a person of skill in the art. Moreover, insertion device operation and IUD insertion procedures can include any number of steps corresponding to a desired IUD position. In addition to the features described below, the insertion devices of the present disclosure include IUD position control features which may be advantageous for insertion of IUDs having a variety of configurations.

The IUD device can also release a time-released drug, active agent, or hormone. The t-shaped IUD 100 would also have an elongate member fabricated of polyethylene equipped with a reservoir adjusted around the elongate member and containing, for example, the hormone levonorgestrel. The reservoir 150 containing the active agent is covered with a polymer membrane that controls the release rate of the drug. The active agent can also include, for example, hormones used for the treatment of menopausal troubles or hormones used for contraception. The IUD is typically provided in sterile packaging together with an insertion device. A membrane can be provided (not shown) which encloses the reservoir 150 and provides for a time-release of the hormone in the reservoir.

As packaged, the T-shaped IUD 100 is positioned at the forward end of the insertion device with the hormone-containing reservoir protected by an outer tube. The IUD arms 120, 122 of the transverse member, on the other hand, are allowed to reside outside the outer tube to prevent creep or shape set of the arms during shipment and storage prior to use. The IUD threads 130, 132 allow the T-shaped IUD 100 to be retracted during removal, are within an interior of the insertion device and extend from a proximal end.

FIG. 2 is a t-shaped IUD 100 with a looped IUD thread 230 positioned within a uterus 30. As would be understood by those skilled in the art, the uterus 30 is accessed via the cervix 32 which is located within the vagina 34. The t-shaped IUD 100 is similar to the IUD shown in FIG. 1 in that it includes an elongated body 110, IUD arms 120, 122 and a connection point 142. However, in this configuration, the looped IUD thread 230 (or string) is provided which forms a loop at the proximal end 10 (the end positioned within the vagina 34). The use of a looped IUD thread 230 provides a mechanism for allowing the IUD thread to be captured for an easier removal of the T-shaped IUD 100.

In some configurations, the IUD threads 130, 132 or looped IUD thread 230 can have a series of knots 160 or beads (not shown) along its length at intervals to assist in the removal process. The one or more knots 160 can be formed by tying each IUD thread in individual knots along the length of the IUD thread. Where beads are used, a small bead can be attached or incorporated into each IUD thread along the length of the IUD thread. The knots or beads would be positioned along the length of the IUD threads at a location along the length of the IUD thread that would be positioned outside the cervix. For the looped IUD thread 230, knots or beads can be provided along a length of the looped IUD thread 230 that is located outside the cervix 32 and within the vagina 34.

### II. Insertion Devices

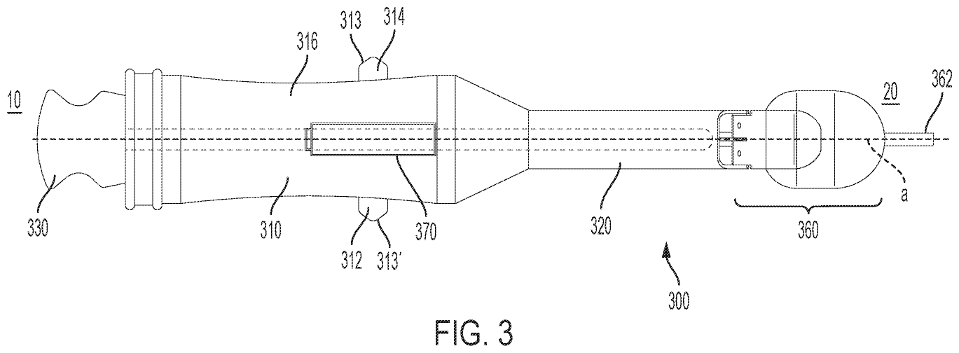

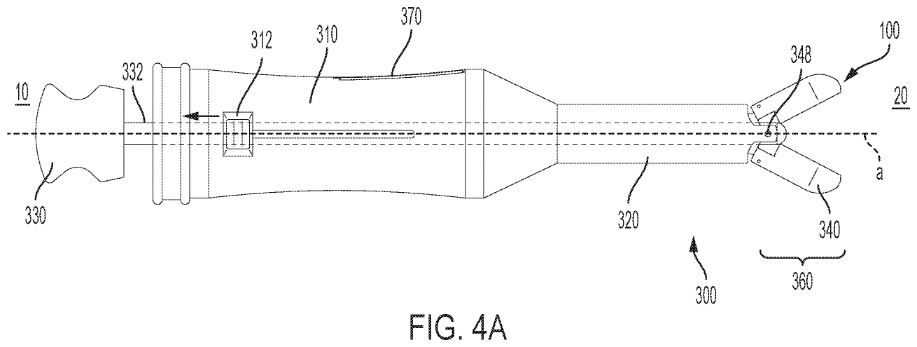

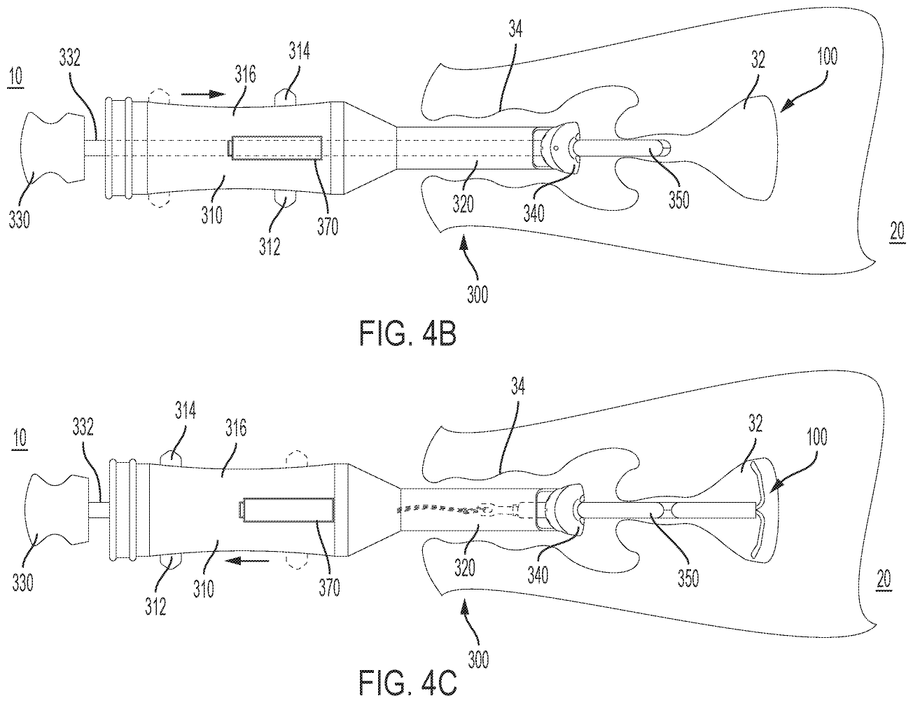

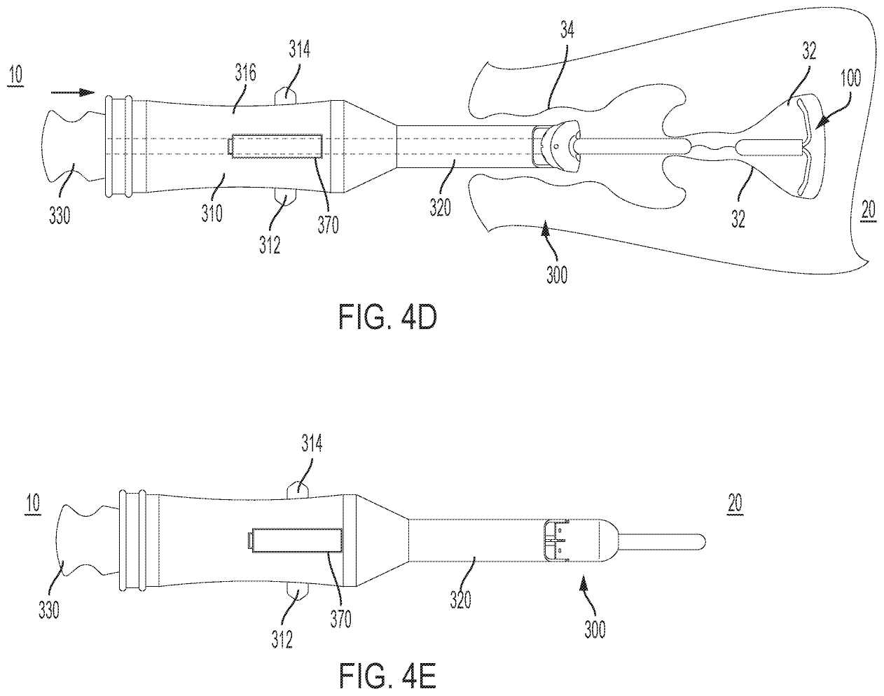

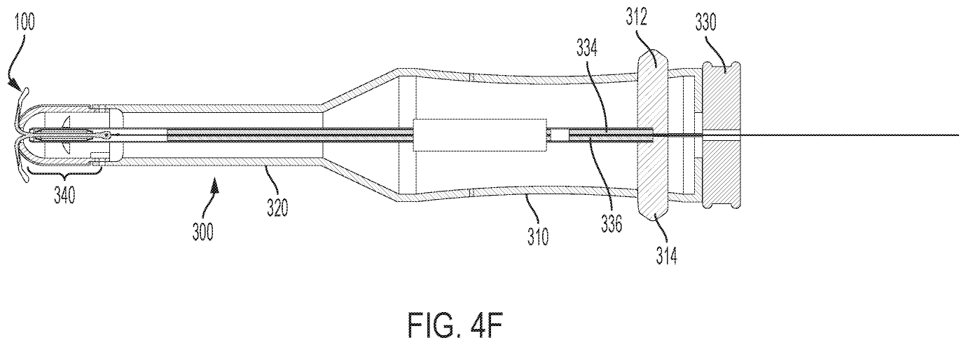

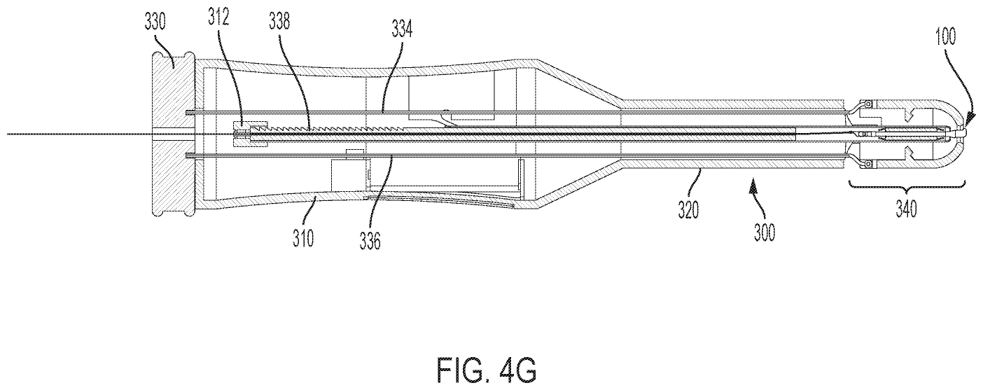

FIG. 3 and FIGS. 4A-G illustrate a self-insertion device 300. A t-shaped IUD 100 is provided (not shown) which resides inside the distal end of the device 300 with its arms 120, 122 open (extended) in FIG. 3. FIGS. 4A-G illustrate the self-insertion device 300 at various stages during an insertion process and a cross-section of the device. Additional information about the operation of self-insertion devices is described below in the method section, Section IV.

The self-insertion device 300 has a handle 310. The handle 310 has a slider 312 The slider 312 can be a bilaterally controlled slider 312, 314 with two separate interfaces exterior to the handle such that it has an interface that a user can engage as illustrated, or a single side controlled slider. Either of the two separate interfaces can control movement of the slider 312 such that the slider moves as a single unit regardless of which side of the device movement of the slider is initiated.

The slider 312 controls the deployment of the t-shaped IUD 100 from the self-insertion device 300. When the slider 312 has a bilateral interface, the slider 312 has an interface positioned on opposite sides of the handle 310 which can be shaped to receive a finger (e.g., thumb) of a user as illustrated. This allows a user to push the slider 312 from either a first surface 313 or a second surface 313′ while achieving the same slider 312 movement making the self-insertion device 300 easily usable by a right-handed or left-handed user. As will be appreciated by those skilled in the art, the slider 312 can be positioned in other locations on the handle 310 without departing from the scope of the disclosure. The slider 312 is also configurable to move in a proximal 10 direction and a distal 20 direction along the length of the handle 310 (i.e., along axis a) in a channel 530 located on the handle 310. Where the slider 312 is bilateral, each interface of the slider 312 had its own channel 530.

The handle 310 can have a curved exterior surface 316, as illustrated, or any other surface configuration that would be useful to improve usability by an end user. For example, multiple curved surfaces corresponding to a finger grip can be provided on the exterior surface. The handle 310 is in communication with a hollow shaft 320. The hollow shaft 320 functions as an insertion tube for use during delivery of the t-shaped IUD 100. A removeable protective cap 360 can be provided. The protective cap 360 can have a tab 362 at the distal end that allows the protective cap 360 to be easily removed. The protective cap 360 can also be a poly bag made from polyethylene which can be sealed onto the tip of the self-insertion device 300 prior to shipment. The tab 362 can be formed during the sealing process and then grabbed by the user to remove the poly bag from the device prior to deployment.

Power to the self-insertion device 300 can be provided by any suitable mechanism including, but not limited to, providing one or more batteries. Where the self-insertion device 300 is powered from an internal source, a compartment can be provided in the handle 310, such as battery compartment 370 which is configured to hold a battery (not shown).

A proximal knob 330 can be provided which is configurable to be pulled in a proximal 10 direction and pushed in a distal 20 direction. The proximal knob 330 engages a first rod 334 and a second rod 336 at a proximal end. The first rod 334 and second rod 336 engage the visualization tool arms at a distal end. Proximal and distal movement of the proximal knob 330 results in opening and closing of the visualization tool arms 340 at the distal 20 end of the device. A hinge 348 can be provided which allows the visualization tool arms to extend or pivot away (opening) from the central axis a, and retract towards (closing) the central axis a in response to the movement of the proximal knob 330.

The proximal movement of the first rod 334 and the second rod 336 via the proximal knob 330 also completes the electrical circuit which provides power to, for example, the light source and the camera. Conversely, distal movement of the first rod 334 and the second rod 336 via the proximal knob 330 opens the electrical circuit which turns-off, for example, the light source and camera.

The first rod 334 and the second rod 336 pass through an interior of the handle 310 and hollow shaft 320 and engage the visualization tool arms 340. The first and second rod pass through the interior handle of the device in a manner which allows free movement in a proximal and distal direction, i.e., clear from the slider which can bisect the device when the slider has a bilateral interface.

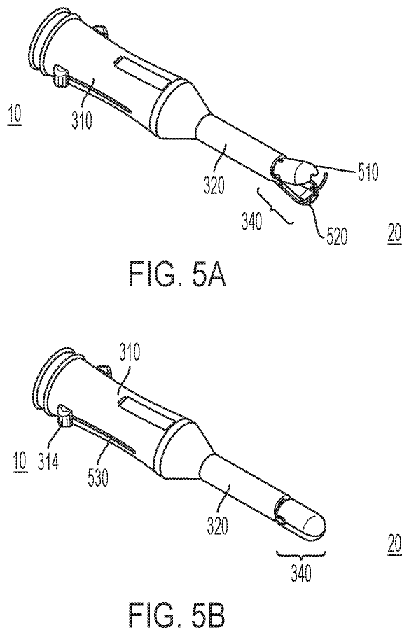

The visualization tool 340, when pushed beyond the distal end 20 of the hollow shaft 320 changes from a configuration where the two or more visualization tool arms 342, 344 are in a closed configuration (e.g., adjacent one another) as shown in FIG. 5B to an open configuration where the visualization tool arms 342, 344 extend away from an axis a as shown in FIG. 5A. The axis a is illustrated bisecting the device along a longitudinal axis.

A plunger can is provided which can be configured with a plurality of angled cuts forming one or more teeth 338 on a side of the plunger. The one or more teeth 338 are positioned within a curved recess 339 along a portion of the length of the plunger. The teeth 338 engage a locking ratchet member 318 projecting from an interior surface of the handle 310. The self-insertion device 300 is further configurable so that the locking ratchet member 318 and teeth 338 do not engage immediately when the slider 312 is moved distally as described in the Method section below. When the slider 312 is moved distally the teeth 338 prevent the plunger from moving proximally once the ratchet mechanism is engaged thereby allowing the IUD to deploy from the inner tube 350.

Once the two or more visualization tool arms 342, 344 extend away from the axis a, the visualization tools located in the distal 20 ends of the visualization tool arms 342, 344 can be activated. Activation of the tools occurs when the electrical switch is engaged. As discussed above, the electrical switch can be configured to engage automatically or semi-automatically with movement of the proximal knob 330. Tools are selected from, for example, light, mirror and camera. Other tools or mechanisms for providing power may be incorporated as desired without departing from the scope of the disclosure. Additionally, more than one tool can be provided on each visualization arm, if desired. Each of the visualization tool arms 342, 344 can have a curved exterior surface. A recess 346 is provided at the distal aperture 345 of the tool arms. The distal aperture 345 and recess 346 allow the IUD arms 120, 122 of the IUD to sit outside the self-insertion device 300 during shipment and prior to deployment. This prevents the IUD from losing the desired t-shape after insertion.

As noted above, the power source is located in the handle 310 and is in communication with any visualization tool that requires power to operate, e.g., light and camera.

In order to stop the procedure prior to completion and after the ratchet mechanism is engaged (the teeth 338 engage the locking ratchet member 318), the user pulls the knob in a distal direction to close the tool arms and then removes the device.

FIGS. 4F-G illustrate cross-sections of a self-insertion device 300 taken at different angles.

Turning now to FIGS. 5A-B, a self-insertion device 300 is depicted from a perspective view with the visualization tool 340 in an open configuration. As illustrated, the visualization tool 340 has two or more arms that extend away from the central axis a. A first visualization arm 342 of the visualization tool 340 is configured to include a camera 510 on an interior surface of the arm and a second visualization arm 344 is configured to include a light source 520. The tools (e.g., camera and light) can be positioned at an angle on the interior surface of the first visualization arm 342 and the second visualization arm 344, e.g., within an angled recess on the interior surface of the visualization arm. Angled mounting of the tools result in the tools facing the area of interest (e.g., cervix of the patent) when the visualization arms extend away from the central axis a. With the tools oriented to point towards the intended visualization target of the cervix 32, the viewer can see the operation of the device. When the first and second visualization arms are drawn together, i.e., towards the central axis a, the interior surfaces of the visualization arms that hold the visualization tools face one another as shown in FIG. 5B.

A channel 530 is provided in the handle 310 which allows the slider 312 to move in a proximal 10 and distal 20 direction within the channel 530. A second channel, not shown, is provided in the handle 310 when the slider 312 has a bilateral interface. The second channel is positioned to allow the second interface of the channel to move in a proximal 10 and distal 20 direction at the same time as the slider 312 moves within the channel 530.

As will be appreciated by those skilled in the art, the camera 510 can be a WiFi enabled camera. A processor 512 or other controller can be provided to communicate with and control the camera 510. A battery, or other suitable power source, can be provided to power the light source 520 and the camera 510.

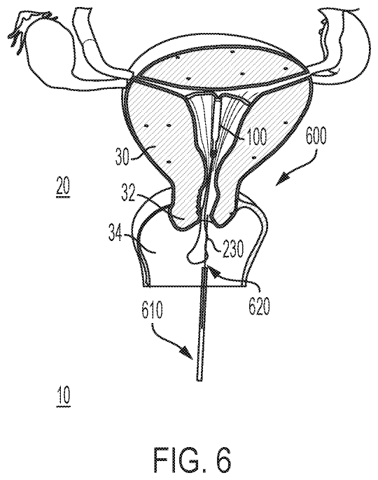

FIG. 6 illustrates a guidewire IUD insertion device 600. The guidewire IUD insertion device 600, has an inserter tube 610 and a guidewire 620. The guidewire 620 is used to access the cervix 32.

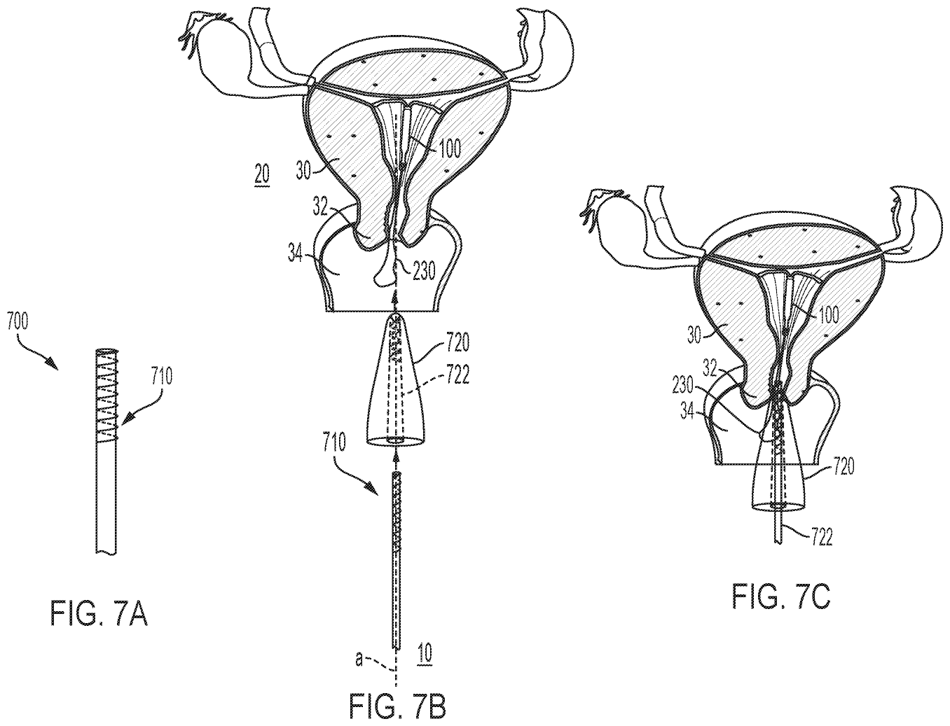

FIGS. 7A-C illustrate a rotational IUD insertion device 700. The rotational IUD insertion device 700 has a threaded silicon tube 710 and a guiding cone 720. The guiding cone 720 has a threaded inner tube 722.

### III. Removal Devices

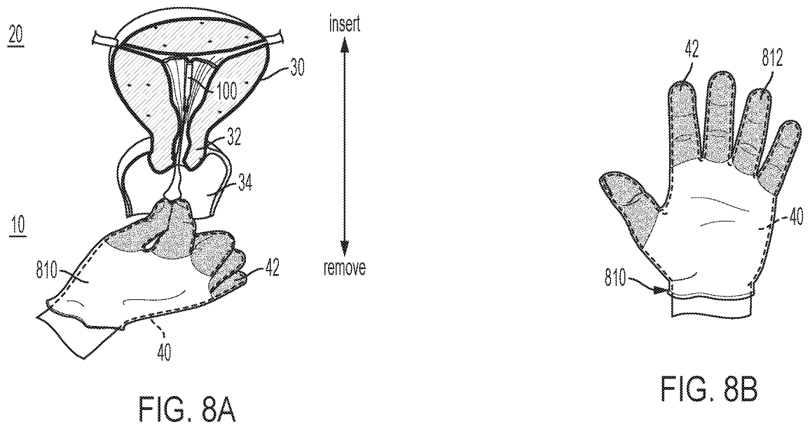

FIGS. 8A-B illustrate a t-shaped IUD 100 positioned within the uterus 30 with the IUD threads 130, 132 positioned within the vagina 34. A hand 40 is shown with a glove 810. The glove 810 has a biologically safe finger coating 812 on one or more of the finger 42 portion of the glove 810. In FIG. 8A, the glove 810 is shown removing the t-shaped IUD 100 by grasping the IUD threads 130, 132 of the t-shaped IUD 100 to remove the IUD. The glove 810 can have a coating or finger covers that attract, for example, the IUD threads 130, 132. FIG. 8B illustrates a hand 40 with the glove 810 and the biologically safe finger coating 812 on the finger portion of the glove 810. The gloves are sized to fit a variety of hand sizes. Sizes are typically determined by measuring the width of the palm below the fingers with most gloves fitting a hand width of from 2 inches (50 mm) to 5.5 inches (140 mm), and sized 2 XS to 3XL.

The glove and coating can be any suitable material. The glove, for example, can be made from rubber, Neoprene™ (polychloroprene) from DuPont, nitrile, or latex. The coating can be made from polyurethane, polyvinyl chloride (PVC), nitrile, foam nitrile, or latex.

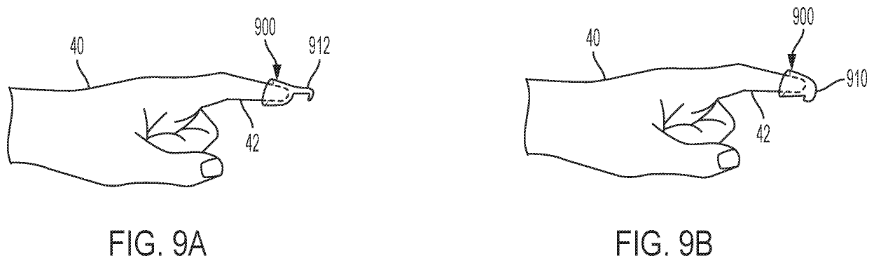

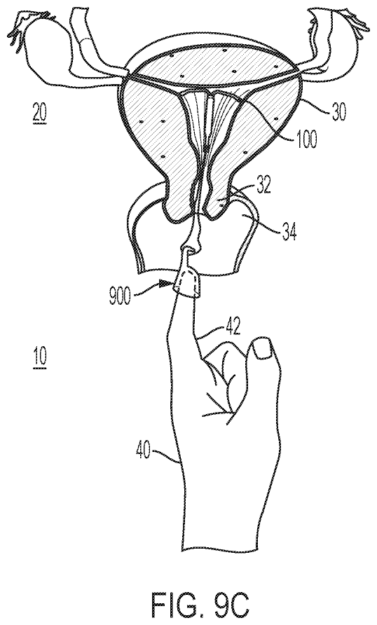

Turning now to FIGS. 9A-C, a finger-cup IUD removal device 900 with a hook 910 for grasping a looped IUD thread is illustrated. A finger 42 of a hand 40 is illustrated with the finger-cup IUD removal device 900. FIG. 9A illustrates a finger-cup IUD removal device 900 with a hook 912 having a sharp curve at the distal tip. FIG. 9B illustrates a finger-cup IUD removal device 900 with a less aggressive hook 910, e.g. a hook with a softer curve. The patient with the IUD inserted or a partner can put the finger-cup IUD removal device 900 on the tip of a finger 42 and reach the finger 42 into the vagina 34. Either form of the hook can be used by the user to catch the looped IUD thread. As shown in FIG. 9C, once the looped IUD thread 930 has been caught by the finger-cup IUD removal device 900, the user pulls on the looped IUD thread 930 (pulling in a proximal direction) to remove the t-shaped IUD 100 from inside the uterus (through the cervix and vagina, outside the body). The size of the finger-cup IUD removal device 900 is sized to fit a variety of finger-tip sizes. Sizes include, for example finger-tip circumferences, typically the index finger or forefinger next to the thumb, of 1.8 inches (46 mm) to 2.75 inches (70 mm), and sized from small to extra-large. The finger cup can be made of any suitable material including, for example, silicone, rubber or Neoprene™.

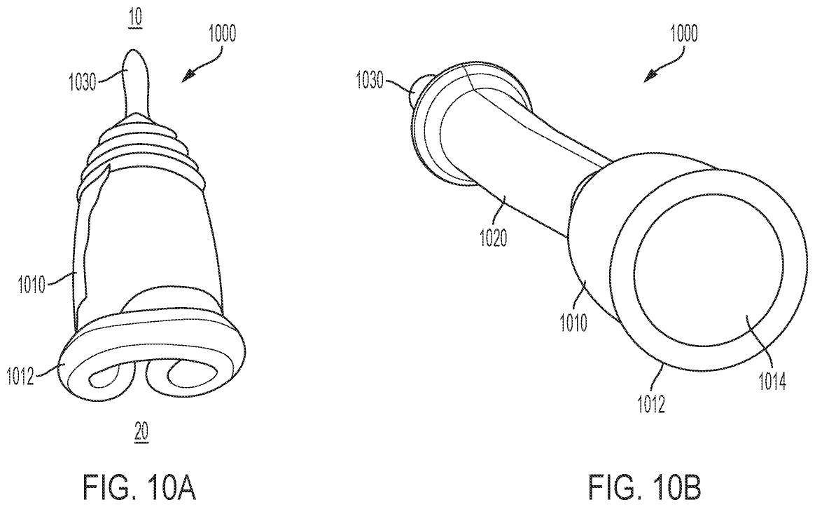

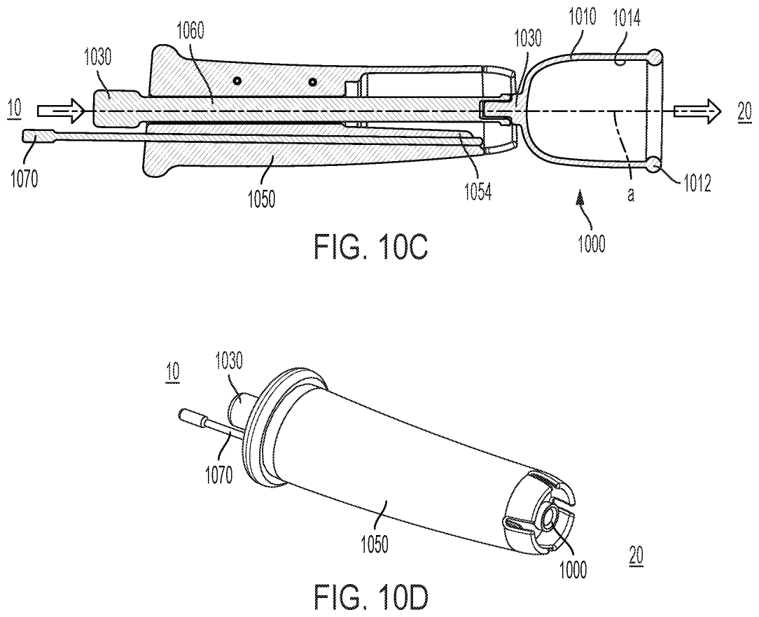

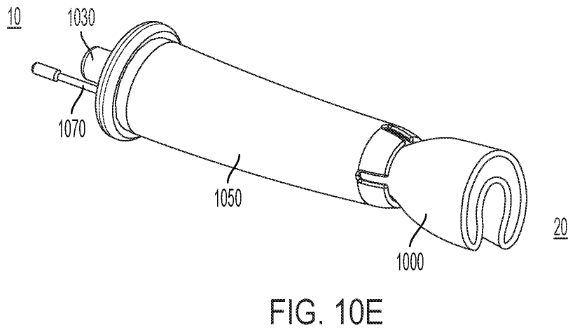

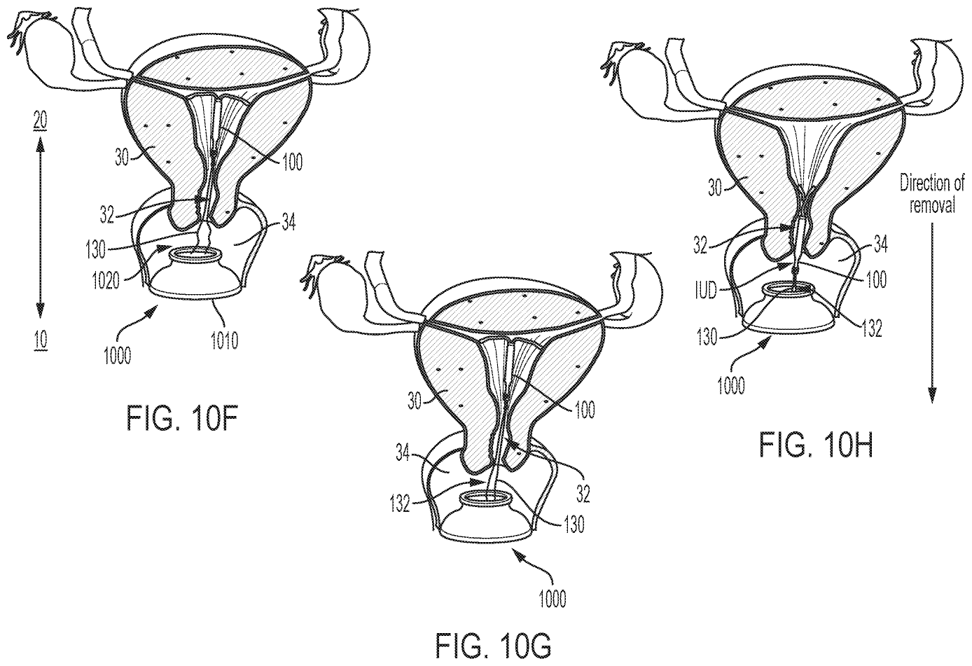

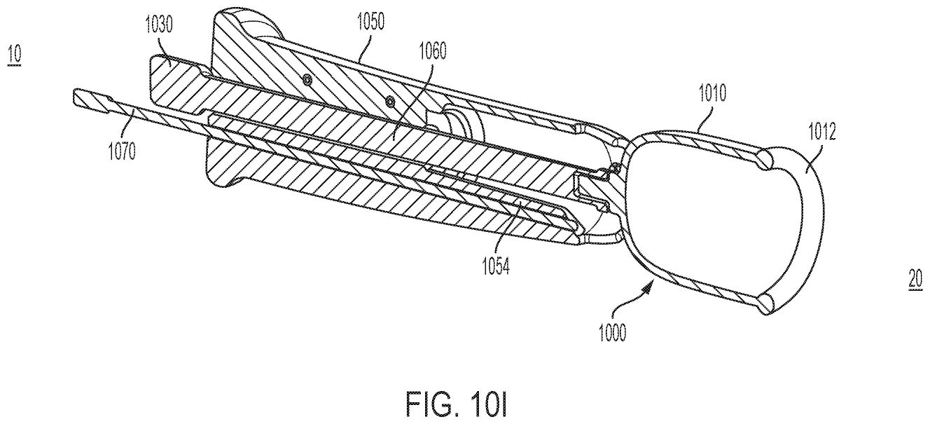

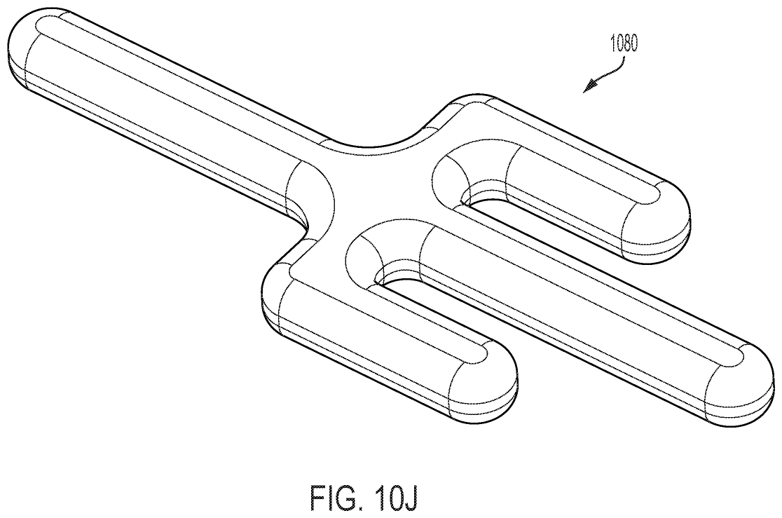

FIGS. 10A-J illustrates an IUD removal device 1000 and tools. The IUD removal device 1000 has a cup 1010 which is rounded and has an internal cavity 1014, exterior walls 1016, and a lip 1012. The cup 1010 is also collapsible to fit within an interior of a removal device delivery tool 1050 during deployment. An additional trident tool 1090 can be provided (shown in FIG. 10J) which is used to facilitate placing the cup 1010 within the interior of an insertion tube 1052 of the removal device delivery tool 1050. The trident tool 1090 can be used instead of the rib 1054, or in addition to the rib 1054, when collapsing the cup 1010 to fit within the removal device delivery tool 1050. In use the cup 1010 applies general suction as the cup 1010 is pulled proximally. This suction is what encourages the IUD to expel. Multiple applications of suction may be required to completely expel the IUD.

A push rod 1060 is provided which has a proximal 10 end that a user can engage and a distal 20 end which engages the cup 1010 of the IUD removal device 1000. A rib 1054 is provided on an interior surface of the insertion tube 1052. The rib 1054 engages an interior surface of the cup 1010 when the cup 1010 is drawn into the central aperture of the insertion tube 1052. When the rib 1054 engages the cup 1010 as the cup 1010 moves proximally, pressure is applied which causes the cup to bend in on itself, lowering the circumference and allowing the collapsed (or bent) cup 1010 to fit within the central aperture of the insertion tube 1052. During shipment the cup 1010 remains outside the insertion tube 1052 to prevent the cup 1010 becoming deformed or losing its optimal shape. A vacuum release rod 1070 is provided which allows the user to release the vacuum on the cup 1010 if the procedure becomes uncomfortable or the patient wants to end the procedure before removing the IUD. The vacuum release rod 1070 can be moved by the user in a distal direction to engage an interior of the cup 1010. As the vacuum release rod 1070 applies pressure to the cup 1010, the shape of the cup 1010 changes (deforms), which causes the cup 1010 to lose vacuum between the cup 1010 and the cervix 32 or vaginal wall.

A proximal connector 1030 is provided which allows the cup 1010 to engage a removal tool 1050 during use. The proximal connector 1030 fits within an interior of the insertion tube 1052. The opening 1020 of the IUD removal device 1000 is configurable to be positioned adjacent the outside of the cervix 32 or in the vaginal canal. The IUD threads 130, 132 (or looped IUD thread) would fall within the opening 1020 of the IUD removal device 1000 when it is positioned adjacent the cervix 32 (as shown in FIGS. 10E-G) and then pressure would be applied to remove the t-shaped IUD 100 from within the uterus 30.

The removal device delivery tool 1050 has a hollow elongated insertion tube 1052 with a central aperture 1056 passing therethrough that receives the IUD removal device 1000 within its interior. A push rod 1060 passes through the central aperture of the hollow elongated insertion tube 1052 and the distal end of the push rod 1060 engages the proximal end of the IUD removal device 1000. Any suitable mechanism for engagement of the distal end of the push rod 1060 and the IUD removal device 1000 can be used without departing from the scope of the disclosure. Suitable mechanisms include male-female connections, snap connections, screw connections, and lock-key connections. The mechanism for engagement of the distal end of the push rod 1060 and the IUD removal device 1000 can be a removable connection such that the end of the plunger 1060 engages the IUD removal device 1000 only during the removal process.

The size of the cup ranges from, for example, a diameter of about 1.6 inches (40 mm) to about 1.8 inches (45 mm), a cup length of from about 1.6 inches (40 mm) to about 2.0 inches (50 mm), a proximal connector 1030 length of from about 0.8 inches (20 mm) to about 1.2 inches (30 mm).

The overall length of the self-removal device with the cup, when the cup is positioned within the self-removal device is from about 100 mm to about 150 mm.

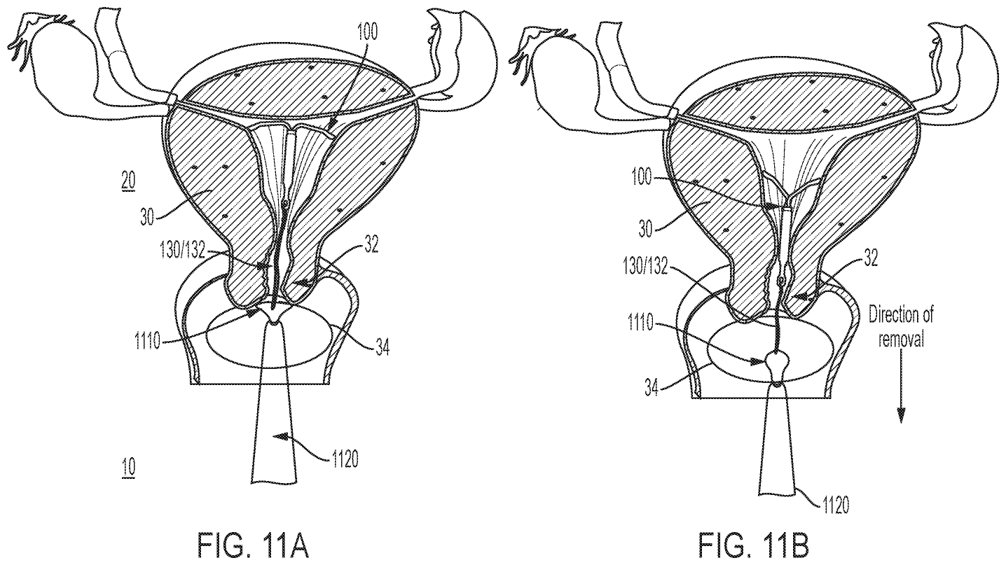

FIGS. 11A-B illustrate another suction cup IUD removal device 1100. As shown in FIGS. 11A-B a cup 1110 is positioned at a distal end 20 of a handle 1120. As will be appreciated by those skilled in the art, the form factor of the handle can take a variety of configurations without departing from the scope of the disclosure.

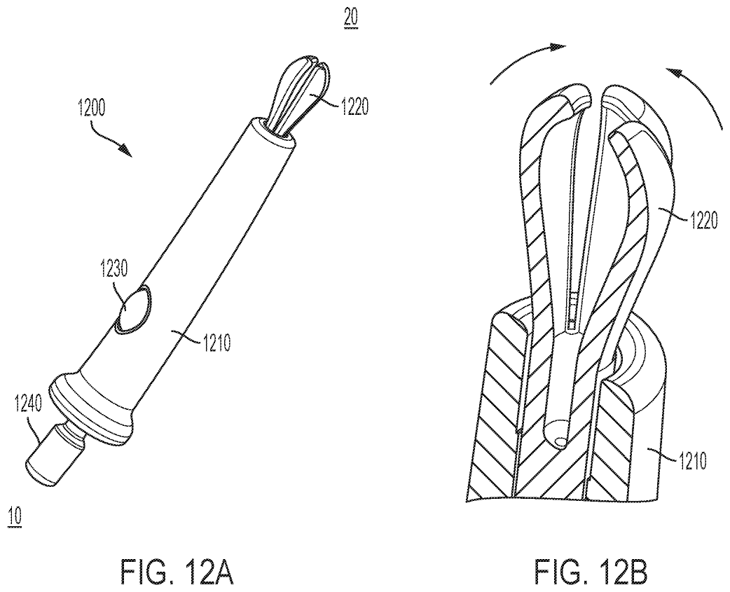

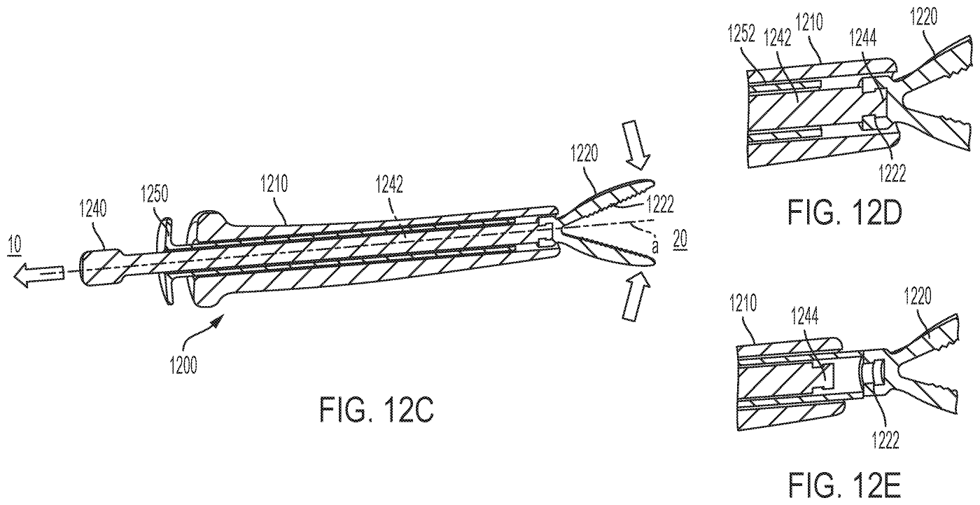

FIGS. 12A-E illustrate a petal-thread grabber IUD removal device 1200. The petal-thread grabber IUD removal device 1200 has an elongated removal tube 1210 with an aperture 1212 therethrough. An elongated shaft 1260 is provided with two or more petals 1220, 1222 positioned at the distal end 20. The elongated shaft 1260 has a user interface 1266 at a proximal end and is configured to pass through the aperture 1212 of the elongated removal tube 1210. In use, the distal end 20 of the elongated removal tube 1210 is pushed into the vagina 34. Once the elongated removal tube 1210 is positioned within the vagina, the two or more petals 1220, 1222 can be extended distally. The two or more petals 1220, 1222 are configurable to have an affinity for the IUD threads 130, 132 (or looped IUD thread). For example, the petals can be made of a material that attracts the threads. The two or more petals 1220, 1222 close-in on the IUD threads 130, 132, locking the IUD threads 130, 132 within the elongated removal tube 1210 via controls (not shown) on the tube handle (not shown). The patient or her partner removes then the t-shaped IUD 100 by pulling the petal-thread grabber IUD removal device 1200 in a proximal direction. The locking controls could be comprised of one or more of the following: sliders, buttons, levers, and knobs that can be pulled, pushed or twisted, etc.

The two or more petals 1220, 1222 can also be configured to have one or more tools, e.g., camera, light etc.

The two or more petals 1220, 1222 can have a one or more ridges on an interior facing surface. The one or more ridges on a first petal can be offset from the one or more ridges on the second petal so that when the two petals are moved towards a central axis, the ridges on a first petal do not engage the ridges on the second petal and the ridges from one petal are adjacent the ridges on the other petal when the two petals are brought together. The ridges can also be made of a material that is different than the material of the petals to provide a transparent surface which does not interfere with a light or camera positioned on the petals during use. Positioning and operation of the tools would be similar to that described above with respect to the self-insertion devices.

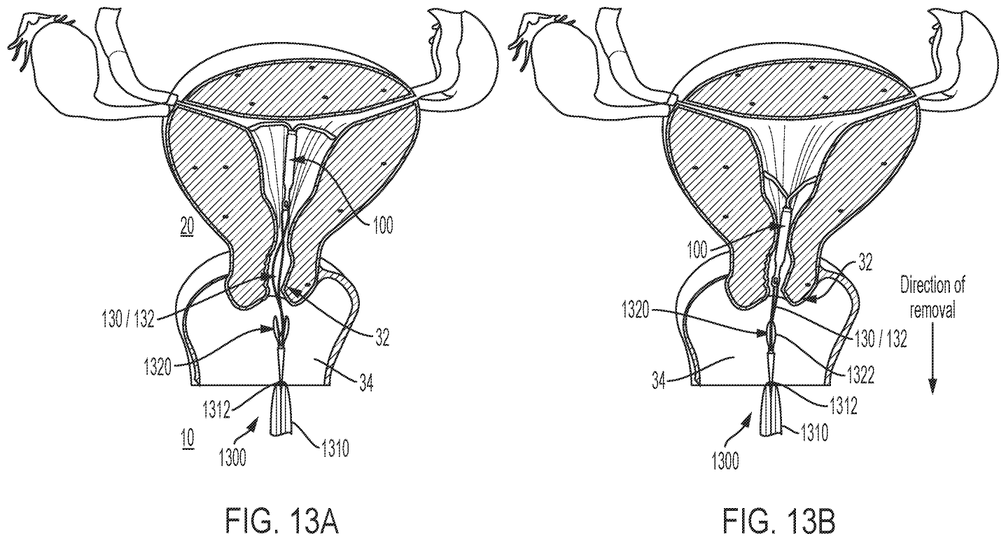

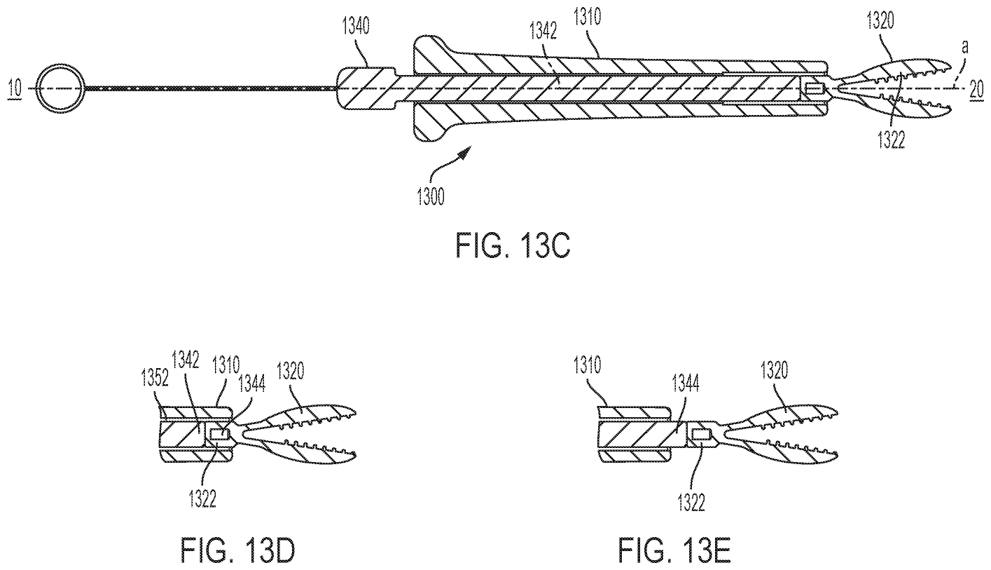

FIGS. 13 A-E illustrate the safety release mechanism in the event the patient or partner wants to stop the procedure after the threads have been engaged.

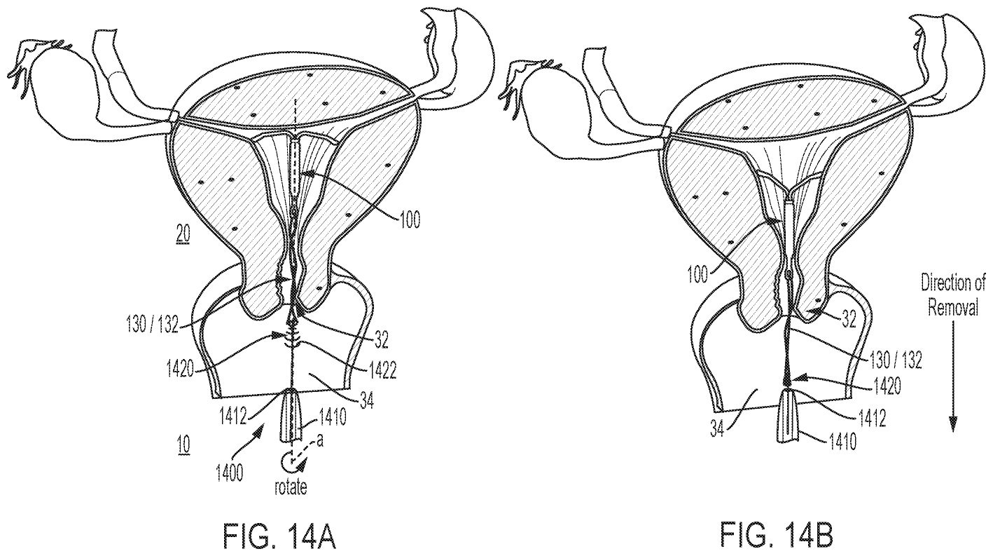

FIGS. 14A-B illustrate a brush thread grabber IUD removal device 1400. The brush thread grabber IUD removal device 1400 has an elongated removal tube 1410 with an aperture 1412 therethrough. A removal tip 1420 is provided at the distal end 20. The removal tip 1420 has a plurality of bristles 1422 extending laterally or substantially laterally from the removal tip 1420. When the brush thread grabber IUD removal device 1400 is inserted into the vagina 34, the brush thread grabber IUD removal device 1400 can be rotated about an axis a to capture the IUD threads 130, 132. Once the IUD threads 130, 132 are captured about the brush thread grabber IUD removal device 1400, the brush thread grabber IUD removal device 1400 can be withdrawn from the vagina 34 by pulling in a proximal direction.

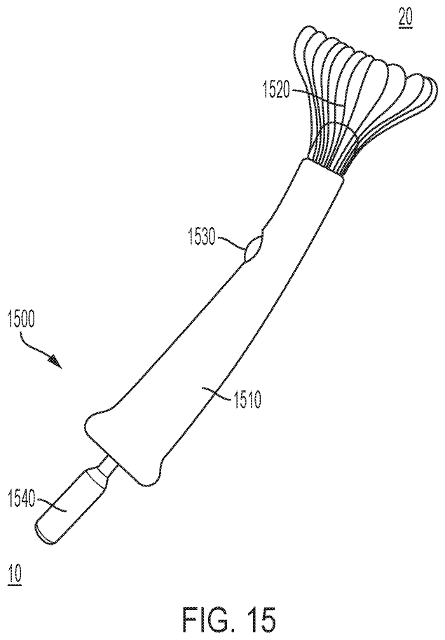

FIG. 15 illustrates an alternative thread grabber IUD removal device 1500. The thread grabber IUD removal device 1500 has an elongated removal tube 1510 with an aperture 1512 therethrough. A removal tip 1520 is provided at the distal end 20. The removal tip 1520 has a plurality of loops 1522 extending from the removal tip 1520. When the thread grabber IUD removal device 1500 is inserted into the vagina 34, the thread grabber IUD removal device 1500 can be rotated about an axis a to capture the IUD threads 130, 132. Once the IUD threads 130, 132 are captured about the thread grabber IUD removal device 1500, the thread grabber IUD removal device 1500 can be withdrawn from the vagina 34 by pulling in a proximal direction.

The plurality of loops 1522 can open or retract when pushed or pulled. The loops 1522 can be made of a flexible plastic material, thin metal wires, or silicone of high Shore hardness. A button 1530 or other suitable control mechanism can be provided which, when pressed, controls the operation of the device by, for example, retracting the distal end of the thread grabber IUD removal device 1500. As will be appreciation, other removal activation interfaces can be used without departing from the scope of the disclosure. For example, the knob 1540 can be turned to cause the distal end to rotate to capture the IUD threads 130, 132.

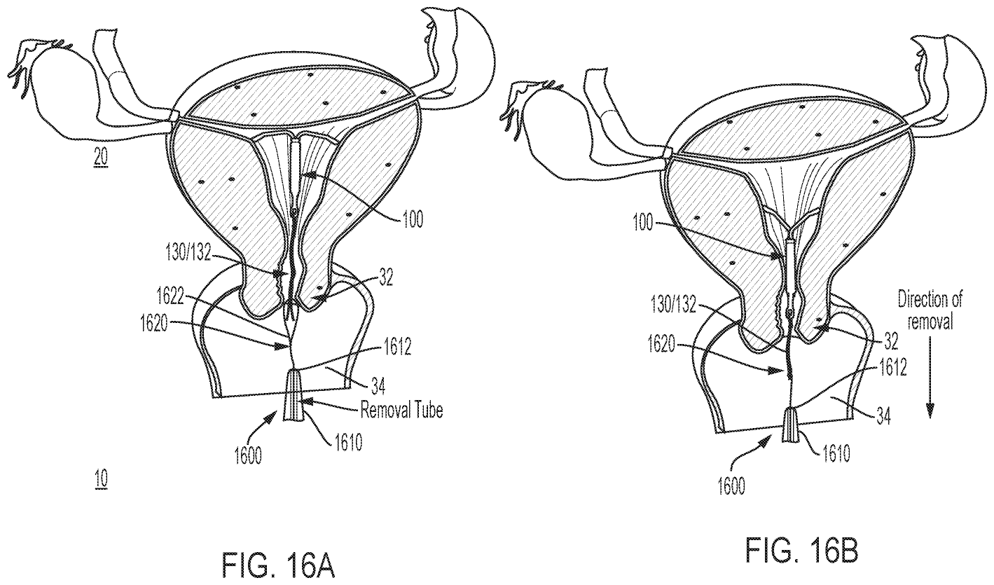

FIGS. 16A-B illustrate a lasso thread grabber IUD removal device 1600. The lasso thread grabber IUD removal device 1600 has an elongated removal tube 1610 with an aperture 1612 therethrough. A lasso removal tip 1620 is provided at the distal end 20. The lasso removal tip 1620 has an affinity for the IUD threads 130, 132. Once the IUD threads 130, 132 are in position, a loop 1622 of the lasso removal tip 1620 cinches around the IUD threads 130, 132. Then the lasso thread grabber IUD removal device 1600 can be pulled proximally 10 to remove the IUD. The lasso thread grabber IUD removal device 1600 can also be used with a looped IUD thread.

### IV. Methods

A. Self-Insertion Devices

The user or her partner downloads and installs an application (“app”). Typically the app will be downloaded to a mobile device or tablet. If the self-insertion device 300 has not been shipped with a battery installed or an external power source is not available, then a battery can be inserted into the battery compartment 370.

The self-insertion device 300, t-shaped IUD 100 and any accessories can be provided in sterile packaging prior to shipping. Additionally, sterile packaging can be provided around the t-shaped IUD 100.

The user pulls on the proximal knob 330 (pulling in a proximal 10 direction) after which the insertion end (distal 20) of the self-insertion device 300 opens, as shown in FIG. 4A, and the t-shaped IUD 100, which had been stored outside the self-insertion device 300 to avoid a long term deformation of the shape, can then be loaded into the hollow shaft 320 of the self-insertion device 300. The user or her partner pulls the t-shaped IUD 100 IUD threads 130, 132 to evenly load the t-shaped IUD 100 into the distal tip of the self-insertion device 300. At this point, if loaded correctly, the t-shaped IUD 100 should not be visible outside the self-insertion device 300. After the t-shaped IUD 100 is correctly loaded the user or her partner can remove and discard the protective cap 360.

Once power is available, the user or her partner tests the light source, such as an LED light source and verifies proper operation of the camera using the application that has been downloaded. The push rod 332 acts as a hard stop for proper positioning of the t-shaped IUD 100 when the IUD is loaded into the device. The plunger floats within the hollow shaft 320, i.e., the plunger is not physically attached to any other component. The plunger is initially constrained by the slider 312.

After loading the t-shaped IUD 100, the self-insertion device 300 is inserted into the vagina 34 until the cervix 32 is reached and the user is no longer able to advance the self-insertion device 300 as shown in FIG. 4B. The user or partner then opens the tip of the self-insertion device as far as comfortable by pulling the knob 330 in a proximal direction. The tools are now powered because movement of the knob 330 in a proximal direction extends the visualization tool arms away from axis a and closes the circuit that provides power from the power source to the tools.

At this point the user or partner should try to visualize the cervix 32. Once the opening to the cervix 32 is observed, the slider 312 is advanced from a proximal position to a distal position within the channel to advance the plunger. A clicking sound should be discernable by the user or partner. The clicking sound is associated with a locking ratchet member 318 engaging the teeth 338 on the plunger within the self-insertion device 300. The plunger and the insertion tube 350 move together when the slider 314 is moved distally. The insertion tube 350 is advanced as far as comfortable. The user or partner then moves the slider 312 in a proximal direction back to the starting position.

The ratchet mechanism will prevent the push rod from moving proximally, and the insertion tube 350 can move independently from the plunger.

The tip of the self-insertion device 300 can be closed. The proximal knob 330 is moved to distally to its starting position to close the tip and bring the two visualization arms together. Then the self-insertion device 300 is slowly removed from the body. The IUD threads 130, 132 should be visible outside the body. An additional removal thread can be provided which engages the IUD threads, e.g., by looping the removal thread through a loop formed by the IUD threads. The removal thread can then be removed by pulling slowly on one loose end.

In another use configuration, a patient or partner inserts a small diameter, guidewire 620 having a soft tip until the guidewire 620 reaches an insertion position. The user would advance the device until resistance is detected. To help avoid perforation, either a marking or physical feature can be provided on the guidewire indicating when, for example, 10 cm has been reached. Markings can be provided to warn the user to stop the advancing the guidewire to prevent perforation. A similar warning mark or feature could be provided on the tube as well. When the proper depth is reached, the woman (or partner) inserts an inserter tube 610 containing a t-shaped IUD (such as the t-shaped IUD 100 shown in FIG. 1) along the guidewire 620 into the cervix. The t-shaped IUD 100 releases from the inserter tube 610 within the cervix 32 and the insertion components are then removed.

Prior to the insertion process, the patient or partner can measure the vaginal depth by inserting a measurement device with markings into the vaginal canal until it touches the cervix and then noting the measurement where the measurement device is at the opening of the canal. The measurement device can be a pliable tubular member with a small radius. The measurement device is sized to exceed the depth of the vagina, e.g. greater than 7 inches (17.7 cm). This measurement can be used to determine correct positioning of the self-insertion device during use.

For a rotation of the rotational IUD insertion device 700, the insertion device is rotated about an axis a in the direction of insertion (e.g., distal direction) versus direct linear force is used to navigate through the cervix 32. Using rotation to advance the rotational IUD insertion device 700 reduces perforation risk.

B. Self-Removal Devices

Using the device of FIG. 8, the patient with the IUD inserted or a partner, or local health promoter, removes the glove from packaging, puts the glove on and reaches inside the vagina 34 to pull on the IUD threads. The glove can have a sticky or tacky coating on one or more fingertip areas which sticks to and/or attracts the IUD threads.

Using the device of FIG. 9, the patient with the IUD inserted or a partner, or local health promoter, removes the finger-cup IUD removal device 900 from packaging, puts the finger-cup IUD removal device 900 on a finger 42 (e.g., index finger) on and reaches inside the vagina 34. The user engages the thread of the t-shaped IUD 100 with the hook 910 of the finger-cup IUD removal device 900 to engage the thread and then removes their finger from the vagina 34 while pulling on the IUD threads to remove the t-shaped IUD 100 from the uterus 30.

Using the IUD removal device of FIGS. 10-11, the patient with the t-shaped IUD 100 inserted, or her partner, would insert the IUD removal device 1000 into the vagina 34 using a removal device delivery tool 1050 (e.g., shown in FIGS. 10C-D and 11A-B). During the insertion process, the cup 1010 can be collapsed and housed within the distal end of the removal tool 1050 (as shown in FIG. 10D). Once the removal device delivery tool 1050 passes into the vagina 34, the cup 1010 can be advanced outside the removal tool 1050. Once the cup 1010 is advanced beyond the distal end of the removal device delivery tool 1050, the cup 1010 changes from a collapsed configuration to an un-collapsed configuration (e.g., opens) and is positioned so that the cup 1010 surrounds a portion of the cervix 32 or can be placed in the vaginal canal slightly away from the cervix 32. A suction force is created between the cup 1010 and the cervix 32 or vaginal wall. Pulling the cup 1010 proximally creates the suction that promotes the IUD to expel from the uterus. If the patient or the partner wishes to stop the procedure after creating suction, the push rod 1060 can be advanced distally to engage the cup 1010 on an exterior surface. The pressure applied by the push rod 1060 then breaks any suction between the cup 1010 and the patient's anatomy.

Using the device from FIGS. 12-13 once the packaging is removed, the petal-thread grabber removal device 1200 is advanced into the vaginal canal. As the petal-thread grabber removal device 1200 approaches the cervix 32, the petals 1220 are opened and then closed on the threads of the IUD. Once the IUD threads are positioned within the petals 1222, the petal-thread grabber removal device 1200 is pulled proximally which should cause the IUD to be withdrawn. Prior to insertion of the petal-thread grabber removal device 1200, the user or partner can measure the depth of the vaginal canal to assess how far the petal-thread grabber removal device 1200 should be advanced. Additionally, tools can be provided in the petals 1222 which, when the petals 1222 are opened activate to provide a visual confirmation of the positioning of the removal device and the IUD threads.

Turning to use of the brush thread grabber IUD removal device 1400 of FIGS. 14A-B, the depth of the vaginal canal can be measured followed by insertion of the brush thread grabber IUD removal device 1400. Once inserted, the user twists the brush thread grabber IUD removal device 1400 to capture the IUD threads, and then pulls the brush thread grabber IUD removal device 1400 in a proximal direction to remove the IUD.

Turning to use of the thread grabber IUD removal device 1500 of FIG. 15, the depth of the vaginal canal can be measured followed by insertion of the thread grabber IUD removal device 1500. Once inserted, the user twists the thread grabber IUD removal device 1500 to capture the IUD threads, and then pulls the thread grabber IUD removal device 1500 in a proximal direction to remove the IUD.

Turning to use of the lasso thread grabber IUD removal device 1600 of FIGS. 16A-B, the depth of the vaginal canal can be measured followed by insertion of the lasso thread grabber IUD removal device 1600. Once inserted, the user tightens the lasso of the lasso thread grabber IUD removal device 1600 to capture the IUD threads, and then pulls the lasso thread grabber IUD removal device 1600 in a proximal direction to remove the IUD.

### V. Kits

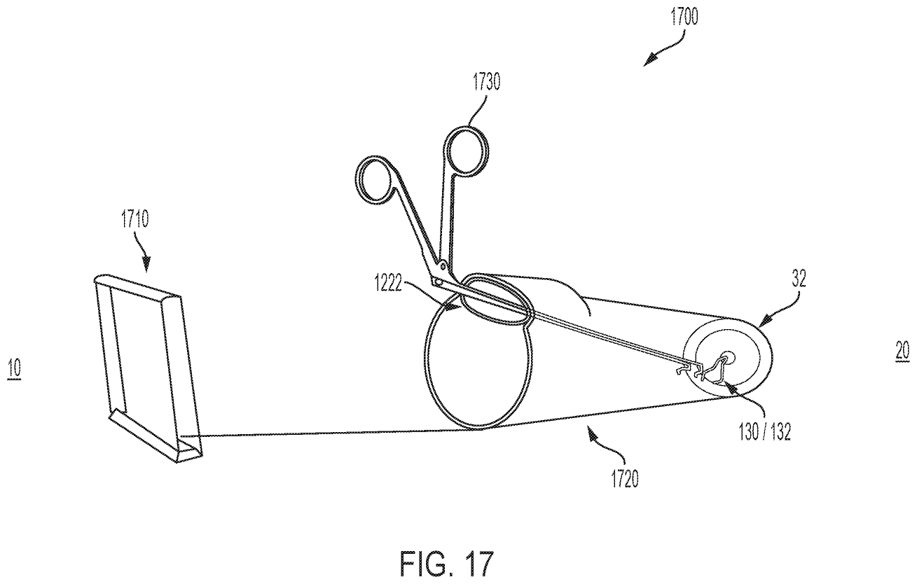

FIG. 17 illustrates a kit 1700 for the IUD insertion devices and IUD removal devices disclosed above. In addition to an insertion or removal device, the kit includes a reflective surface or a camera 1710. The reflective surface or camera can be particularly useful for configurations of an insertion device or removal device that do not include tools.

A self-illuminating cone assembly 1720 is provided. Forceps 1730 and a forceps guide 1732 can also be provided. The self-illuminating cone assembly 1720 can be similar to a self-contained amnioscope which provides a shadow-free light source and allows access to the vagina 34. The forceps 1730 have a 90 degree bend and pass through a forceps guide 1722 in the self-illuminating cone assembly 1720 to access the cervix 32. The forceps 1730 grab the IUD threads. The reflective surface 1710, such as a mirror or phone mount attached to the self-illuminating cone assembly 1720, provides visual feedback during the removal procedure. A vaginal canal measurement device can be provided to assist in determining the depth of the vaginal canal. A software application (“app”) can also be provided for a mobile device that allows a patient (or partner) to upload a picture of the removed IUD to provide digital confirmation of successful removal.

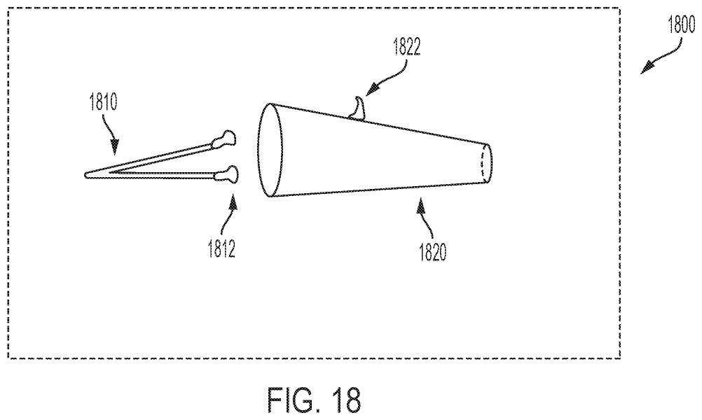

FIG. 18 illustrates another kit 1800 for removing an IUD. The disposable kit 1800 has forceps 1810 where the forceps ends 1812 are covered in cotton. A visualization cone 1820 can be included which has a positioning hook 1822. Upon inserting the visualization cone 1820, the user holds the visualization cone in place using the ergonomic finger hold (positioning hook 1822). A partner can then reach into the vagina with the forceps 1810. The forceps ends 1812 are covered in cotton and grasp onto the IUD strings with ease. The partner secures the strings with the forceps 1810 and pulls in a proximal direction to remove the IUD.

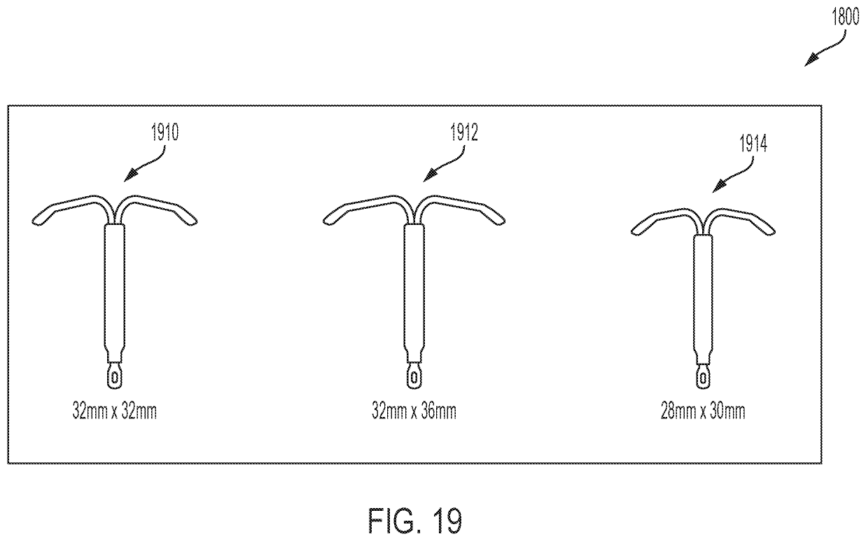

FIG. 19 illustrates a self-removal verification card 1900. The self-removal verification card can include images 1910, 1912, 1914 of a plurality of potential IUD shapes and sizes (shown as t-shaped IUDS of sizes 32 mm×32 mm 1910, 32 mm×36 mm 1912, and 28 mm×30 mm 1914). The images can be different any combination of shapes and/or sizes of IUDs. Upon removal of the IUD, the user places the IUD on the verification card. Once the removed IUD overlaps fully with one of the images provided on the verification card, confirmation of full removal of the IUD is achieved.

As discussed above, an app can be provided as part of a kit for inserting or removing an IUD. The app can include a connection to a healthcare provider and a process for securely uploading one or more images related to the procedure. The app would typically be installed on a mobile device having a camera function.

For removal, the patient (or partner) can upload a photograph of the removed IUD for a healthcare provider to review to confirm that the IUD was properly and completely removed. Additional images mat be provided as needed or requested.

For insertion, a user (or partner) may take one or more images during the insertion process and load those images into the secure app to allow a remote healthcare provider to determine whether there are obvious complications to the insertion process.

As will be appreciated by those skilled in the art, the applications and methods according to aspects of the disclosed subject matter may utilize a variety of computer and computing systems, communications devices, networks and/or digital/logic devices for operation. Each may, in turn, be configurable to utilize a suitable computing device that can be manufactured with, loaded with and/or fetch from some storage device, and then execute instructions that cause the computing device to perform a method according to aspects of the disclosed subject matter.

A computing device can include, without limitation, a mobile user device such as a mobile phone, a smart phone, a personal digital assistant (“PDA”), such as an iPhone®, a tablet, a laptop, a smart watch, and the like. In at least some configurations, a user can execute a browser application over a network, such as the internet, to view and interact with digital content provided by a healthcare provider, such as screen displays. A display includes, for example, an interface that allows a visual presentation of data from a computing device. Access could be over or partially over other forms of computing and/or communication networks. A user may access a web browser, for example, to provide access to applications and data and other content located on a website or a webpage of a website.

A suitable computing device may include a processor to perform logical and other computing operations, for example, a stand-alone central processing unit (“CPU”), or hard-wired logic circuit as in a microcontroller, or a combination of both, and may execute instructions according to its operating system and the instructions to perform the steps of the method, or elements of the process. The user's computing device may be part of a network of computing devices and the methods of the disclosed subject matter may be performed by different computing devices associated with the network, perhaps in different physical locations, cooperating or otherwise interacting to perform the disclosed method. For example, a user's portable computing device may run an application alone or in conjunction with a remote computing device, such as a server on the internet or on an intranet. For purposes of the present application, the term “computing device” includes any and all of the above discussed logic circuitry, communications devices and digital processing capabilities or combinations of these.

Certain embodiments of the disclosed subject matter may be described for illustrative purposes as steps of a method that may be executed on a computing device executing software, and illustrated, by way of example only, as a block diagram of a process flow. Such may also be considered as a software flow chart. Such block diagrams and like operational illustrations of a method performed or the operation of a computing device and any combination of blocks in a block diagram, can illustrate, as examples, software program code/instructions that can be provided to the computing device or at least abbreviated statements of the functionalities and operations performed by the computing device in executing the instructions. Some possible alternate implementation may involve the function, functionalities and operations noted in the blocks of a block diagram occurring out of the order noted in the block diagram, including occurring simultaneously or nearly so, or in another order or not occurring at all. Aspects of the disclosed subject matter may be implemented in parallel or seriatim in hardware, firmware, software or any combination(s) of these, co-located or remotely located, at least in part, from each other, for example, in arrays or networks of computing devices, over interconnected networks, including the internet, and the like.

As will be appreciated by those skilled in the art, in some embodiments, any suitable computer readable media can be used for storing instructions for performing the functions and/or processes herein. For example, in some embodiments, computer readable media can be transitory or non-transitory. For example, non-transitory computer readable media can include media such as magnetic media (e.g., hard disks, floppy disks, and/or any other suitable magnetic media), optical media (e.g., compact discs, digital video discs, Blu-ray discs, and/or any other suitable optical media), semiconductor media (e.g., flash memory, erasable programmable read-only memory (EPROM), electrically erasable programmable read-only memory (EEPROM), and/or any other suitable semiconductor media), any suitable media that is not fleeting or devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An IUD self-insertion device comprising:
an elongated handle with a knob at a proximal end and a control slider moveable in a channel along a length of the handle;
a hollow shaft positioned at a distal end of the elongated handle;
a plunger positioned within the handle and the hollow shaft having a plurality of teeth along at least a portion of an outer surface of the plunger;
two or more arms positioned at a distal end of the hollow shaft wherein the arms are rotatable away from a central axis of the hollow shaft;
a ratchet member positioned on an interior surface of the handle configured to engage the plurality of teeth along the outer surface of the plunger;
one or more tools positioned on an interior surface of at least one of the arms; and
a power source in communication with the one or more tools.

2. The IUD self-insertion device of claim 1, wherein the control slider is a bilaterally controllable slider with a first slider interface moveable within a first channel and a second slider interface moveable within a second channel.

3. The IUD self-insertion device of claim 1, wherein the elongated handle has a curved exterior surface.

4. The IUD self-insertion device of claim 1, wherein the knob is configurable to move in a proximal and distal direction.

5. The IUD self-insertion device of claim 1, wherein the plunger is configured to float within the interior of the self-insertion device.

6. The IUD self-insertion device of claim 1, wherein the tools are selected from a light source and a camera.

7. The IUD self-insertion device of claim 1, wherein the visual tool arms have a curved recess in an outer surface at the distal end that holds arms of an IUD prior to loading the IUD into the insertion device wherein the IUD has a t-shape and comprises an elongated member and a pair of IUD arms.

8. The IUD self-insertion device of claim 1, wherein the elongated member of the IUD comprises a core part around which a jacket-like polymeric reservoir containing an active agent has been fitted.

9. The IUD self-insertion device of claim 8, wherein the active agent is a hormone used for the treatment of menopausal troubles or for contraception.

10. An IUD self-removal device comprising:
an elongated handle with a push rod moveable within an interior of the handle;
a cup comprising a wall with an inner wall surface defining a cavity and an outer wall surface, an open end and a closed end, a stem extending from an exterior location on the wall opposite the open end wherein the stem is configurable to engage a distal end of the push rod.

11. The IUD self-removal device of claim 10, further comprising a vacuum release rod.

12. The IUD self-removal device of claim 10, further comprising a rib on an interior surface of the elongated handle.

13. An IUD self-removal device comprising:
an elongated handle forming an elongated shaft;
a push rod positioned within the elongated handle having a connector at a distal end of the push rod; and
a grabber comprising two or more petals positioned at a distal end of the push rod wherein the petals are rotatable away from a central axis of the push rod wherein an interior surface of the two or more petals have ridges.

14. The IUD self-removal device of claim 13, further comprising one or more tools positioned on an interior surface of at least one of the petals.

15. The IUD self-removal device of claim 14, further comprising a power source in communication with the one or more tools.

16. The IUD self-removal device of claim 14, wherein the tools are selected from a light source and a camera.

17. The IUD self-removal device of claim 13, wherein the elongated handle has a curved exterior surface.

18. The IUD self-removal device of claim 13, wherein the push rod is configurable to move in a proximal and distal direction.

19. An IUD self-removal device comprising:
an elongated handle with a push rod having a distal end and a proximal end wherein the push rod is moveable in a distal direction and a proximal direction within an interior of the handle;
at least one of
bristles positioned at the distal end of the push rod, a plurality of threads positioned at the distal end of the push rod, a lasso positioned at the distal end of the push rod, and a hook positioned at the distal end of the push rod.

20. The IUD self-removal device of claim 19, further comprising one or more tools positioned on a surface of the one or more petals.

21. The IUD self-removal device of claim 19, wherein the tools are selected from a light source and a camera.

22. A kit comprising:
at least one of a self-insertion device with an IUD and a self-removal device; and
at least one of a vaginal cavity depth measurement device; a battery; forceps; a visualization cone; and a self-removal verification card.

23. The kit of claim 22, wherein the visualization cone has a forceps guide positioned on a surface.

24. The kit of claim 22, wherein the visualization cone is self-illuminating.

25. A method of inserting an IUD into a patient comprising the steps of:
providing a self-insertion device comprising an elongated handle with a knob at a proximal end and a control slider moveable in a channel along a length of the handle, a hollow shaft positioned at a distal end of the elongated handle, a plunger positioned within the handle and the hollow shaft having a plurality of teeth along at least a portion of an outer surface of the plunger, two or more arms positioned at a distal end of the hollow shaft wherein the arms are rotatable away from a central axis of the hollow shaft, a ratchet member positioned on an interior surface of the handle configured to engage the plurality of teeth along the outer surface of the plunger, one or more tools positioned on an interior surface of at least one of the arms, and a power source in communication with the one or more tools;
inserting the self-insertion device into a vaginal cavity;
pulling the knob of the self-insertion device in a distal direction;
viewing a cervix;
advancing the inner delivery tube into the cervix;
releasing the IUD from the self-insertion device; and
removing the self-insertion device.

26. The method of inserting an IUD into a patient of claim 25, further comprising the step of:
measuring a depth of the vaginal cavity.

27. The method of inserting an IUD into a patient of claim 25, further comprising the steps of:
acquiring an image of the cervix; and
transmitting the image of the cervix to a location remote from the patient.

28. A method of removing an inserted IUD from a patient comprising the steps of:
providing a self-removal device comprising an elongated handle with a push rod moveable within an interior of the handle, a cup comprising a wall with an inner wall surface defining a cavity and an outer wall surface, an open end and a closed end, a stem extending from an exterior location on the wall opposite the open end wherein the stem is configurable to engage a distal end of the push rod;
inserting the self-removal device into a vaginal cavity;
positioning the cup near the cervix;
creating a suction around an IUD removal thread;
pulling the cup in a proximal direction; and
removing the cup and self-insertion device.

29. The method of removing the inserted IUD from the patient of claim 28, wherein the further self-removal device further comprises a vacuum release rod.

30. The method of removing the IUD from the patient of claim 28, further comprising:
measuring a depth of the vaginal cavity.

31. The method of removing the inserted IUD from the patient of claim 28, further comprising:
acquiring an image of the cervix; and
transmitting the image of the cervix to a location remote from the patient.

32. A method of removing an inserted IUD from a patient comprising the steps of:
providing a self-removal device comprising an elongated handle forming an elongated shaft, a push rod positioned within the elongated handle having a connector at a distal end of the push rod, and a grabber comprising two or more petals positioned at a distal end of the push rod wherein the petals are rotatable away from a central axis of the push rod wherein an interior surface of the two or more petals have ridges;
inserting the self-removal device into a vaginal cavity;
positioning the petals near the cervix;
pushing the push rod in a distal direction to extend the petals away from a central axis;
pulling the push rod in a proximal direction to retract the petals towards the central axis to engage the IUD threads; and
removing the self-removal device.

33. The method of removing the IUD from the patient of claim 32, further comprising the step of:
measuring a depth of the vaginal cavity.

34. The method of removing the inserted IUD from the patient of claim 32, further comprising the steps of:
acquiring an image of the cervix; and
transmitting the image of the cervix to a location remote from the patient.
